(19) United States
(12) Patent Application Publication
(10) Pub. No.: US 2024/0027633 A1
(43) Pub. Date: Jan. 25, 2024

(54) DOSE MONITOR FOR FLASH RADIOTHERAPY

(71) Applicant: UNIVERSITY OF UTAH RESEARCH FOUNDATION, Salt Lake City, UT (US)

(72) Inventors: Edward CAZALAS, Salt Lake City, UT (US); Codey OLSON, Salt Lake City, UT (US); Geoff NELSON, Salt Lake City, UT (US)

(21) Appl. No.: 17/993,415

(22) Filed: Nov. 23, 2022

Related U.S. Application Data
(60) Provisional application No. 63/391,612, filed on Jul. 22, 2022.

(51) Int. Cl. G01T 1/20; G01T 3/06

(52) U.S. Cl. CPC G01T 1/201; G01T 3/06

(57) ABSTRACT

This disclosure is directed to a dosimeter that may be utilized in FLASH radiotherapy (FLASH-RT) applications. The dosimeter is capable of responding on small time scales while simultaneously maintaining a well-defined response with ultra-high dose rates. The dosimeter is capable of real-time and in-situ control and monitoring of beam delivery, which beneficially enhances the safety and accuracy of FLASH-RT treatments.

### CROSS-REFERENCE TO RELATED APPLICATIONS

This application claims priority to and the benefit of U.S. Provisional Patent Application No. 63/391,612, filed Jul. 22, 2022 and titled Dose Monitor for Flash Radiotherapy, the entirety of which is incorporated herein by reference.

### BACKGROUND

### Technical Field

This disclosure is directed to a dosimeter that may be utilized in FLASH radiotherapy (FLASH-RT) applications.

### Related Technology

Over the past decade, an increase in interest of a new radiotherapy method termed “FLASH” radiotherapy (FLASH-RT) has significantly altered the potential of radiation-based cancer treatment. Studies of the FLASH effect indicate that the delivery of radiation at ultra-high rates can increase the survival of healthy tissue surrounding a tumor site while simultaneously maintaining a high probability of tumor death.

As compared to conventional radiotherapy (RT) methods, FLASH-RT requires more advanced delivery monitoring and control systems. Currently, nearly all RT treatments are monitored in real-time by ionization (ion) chambers. However, at the ultra-high dose rates required for the FLASH effect, these detectors are subject to extreme charge recombination where the response is severely impacted with increased dose-rates.

Accordingly, there is an ongoing need for a dosimetry monitoring system with the capability of responding on extremely small time scales while simultaneously maintaining a well-defined response with ultra-high dose rates.

### SUMMARY

This disclosure is directed to a FLASH-RT dosimeter capable of real-time and in-situ control and monitoring of beam delivery, which beneficially enhances the safety and accuracy of FLASH-RT treatments.

In one embodiment, a dosimeter configured for use in FLASH-RT comprises: a scintillator configured for positioning within a beam path of a proton beam generator; a plurality of fiberoptic lines each having a first end and a second end, the first ends being optically coupled to the scintillator; a photomultiplier to which the second ends of the plurality of fiberoptic lines are optically coupled; and a controller to which the photomultiplier is communicatively coupled, the controller being configured for communication with a beam controller of the proton beam generator.

In some embodiments, the scintillator comprises a plurality of scintillating fiberoptic lines, which may optionally be arranged in a cross-hatched configuration. Each scintillating fiberoptic line may be coupled to a separate fiberoptic line for transmission of light signals from the scintillator to the photomultiplier. In some embodiments, the scintillator is a flat sheet. The flat sheet may have a thickness of 1 mm to 5 mm.

In some embodiments, the fiberoptic lines are coupled to a side edge of the scintillator and extend therefrom (i.e., extend laterally therefrom away from the beam path). In some embodiments, the dosimeter includes one or more light guides associated with the scintillator to guide light signals from the scintillator material to the fiberoptic lines.

In some embodiments, the dosimeter includes a converter communicatively coupled to the photomultiplier and the controller and configured to adjust a bias voltage of the photomultiplier and/or to function as an amplifier.

In some embodiments, the scintillator includes a BC-420 scintillator material and/or similar scintillator material, such as one that provides one or more of: a rise time of 0.5 ns; a decay time of 1.5 ns; a maximum emission peak of 391 nm; an emission level of 64% anthracene; a density of 1.032 g/cc; or a refractive index of 1.58, or one or more similar values within ±10%, or ±7.5%, or ±5%, or ±2.5% of the foregoing.

In some embodiments, the scintillator comprises an aromatic plastic, such as polyvinyltoluene (PVT) and/or polystyrene (PS). In some embodiments, the photomultiplier comprises a silicon photomultiplier (SiPM). In some embodiments, the controller comprises a field programmable gate array (FPGA).

In some embodiments, a FLASH-RT system comprises: a proton beam generator; and a dosimeter as disclosed herein, disposed within a beam path of the proton beam generator.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1A illustrates various definitions utilized to quantify dose-rate during RT, and FIG. 1B compares common values in conventional RT to FLASH-RT, as described in Ashraf et al., “Dosimetry for FLASH Radiotherapy: A Review of Tools and the Role of Radioluminescence and Cherenkov Emission” Front. Phys., 21 Aug. 2020;

FIGS. 2A and 2B are schematics of example dosimeters;

FIG. 3 is an emission spectrum of an example scintillator material;

FIGS. 4A-4D show normalized energy deposition within a tissue phantom versus depth into tissue, showing variations in the Bragg peak location for BC-420 and YAP:Ce scintillators with a thickness of 5 mm, in comparison to no scintillator, at four primary proton energies (50, 100, 150, 200 MeV); and

FIG. 5 is a curve-fitting graph showing a well-defined response between scintillator energy deposit and the total dose to a tissue phantom for an example scintillator.

### DETAILED DESCRIPTION

### Overview of FLASH-RT

The major difference between FLASH-RT and conventional RT is the targeted dose-rate utilized in treatment. In conventional RT, treatments are generally segmented over multiple sessions, each on the order of a few minutes and recurring over multiple hours or days. During these irradiations, a dose between 10-40 Gy (J kg⁻¹) is typically delivered (dependent on the treatment plan), where the average dose-rates are maintained around 1-2 Gy min⁻¹. In contrast, while the exact parameters and thresholds for FLASH are still being studied, it is generally believed that average dose-rates above 40 Gy s⁻¹ are sufficient to produce the tissue sparing effect.

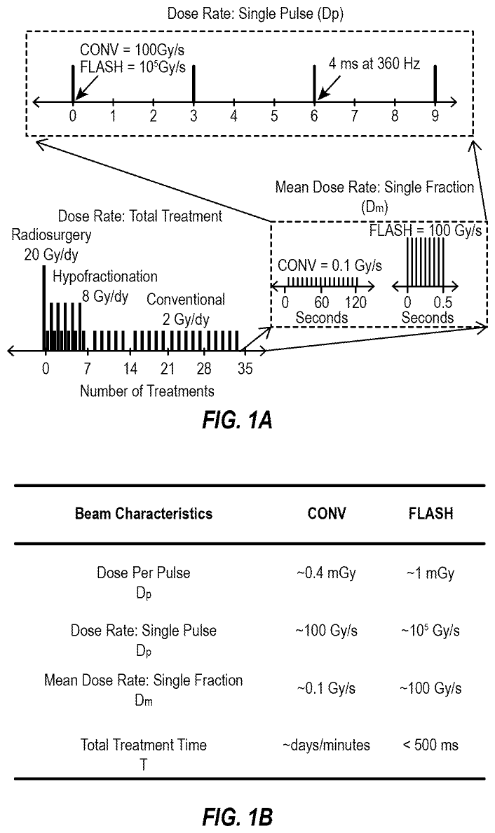

Delivery rates can be quantified utilizing definitions of Dose Per Pulse (DPP), Single Pulse Dose Rate (SPDR), and Mean Dose Rate (MDR). These definitions are shown in FIG. 1A, with comparisons of these beam characteristics shown for conventional RT and FLASH-RT shown in FIG. 1B. Here, a major hurdle for FLASH can be seen, as SPDRs can be upwards of 10⁵ Gy s⁻¹, which is three orders of magnitude higher than that of conventional treatments and MDRs. Given the drastically higher delivery rates, the total delivery time is necessarily much shorter—often an entire treatment is less than 500 ms—and thus much finer temporal resolutions are required to allow for adequate monitoring.

The most utilized method of beam monitoring during conventional RT are ion chambers. At the comparatively low dose-rates of conventional RT, ion chambers respond linearly with increased dose-rate, providing an affordable and easy to utilize instrument for direct beam monitoring. However, at the high dose-rates of FLASH, and particularly the high Single Pulse Dose Rates of −10⁵ Gy s⁻¹, these chambers undergo charge effects (including recombination and electric field reduction) and lose this linearity. Some works have studied this effect and introduced correction factors to account for this effect, yet results remain inadequate for most FLASH applications.

Some other detection methods have proven to be dose-rate independent, maintaining linear responses with increasing dose rates and allowing for the high SPDRs exhibited, but fail for other reasons. Gafchromic films, for example, have displayed this characteristic and are a common tool for current beam monitoring and calibrations to provide very accurate spatial measurements of the beam as well. Chemical based dosimeters utilizing ferrous oxides or methyl viologen have also shown a similar rate-independence. However, such methods cannot be performed in-situ, often requiring 24-hour post-irradiation counting. In general, chemical dosimeters and films are valuable tools for conventional RT, but fall short of the desired characteristics and are unsuitable for any real-time FLASH applications.

Diffenderfer et. Al. (Design, Implementation, and in Vivo Validation of a Novel Proton FLASH Radiation Therapy System, Int. J. Radiat. Oncol. Biol. Phys. 106 (2020) 440-448) have shown that the use of a sodium iodide (NaI) scintillator and conventional ion chamber can allow for real-time pulse monitoring. In their study, they show that the use of NaI can allow for the measurements of prompt gamma-ray emissions from proton interactions at the exit of the beam gantry, prior to the patient. In conjunction with the ion chamber, absorbed dose and rate calculations of individual beam pulses can be performed. However, the use of the ionization chamber significantly inhibits the response time. The authors report a beam on/off latency of roughly 150 microseconds, likely attributed to the long decay time of NaI.

Bourguin et. Al. (calorimeter for Real-Time Dosimetry of Pulsed Ultra-High Dose Rate Electron Beams, Front. Phys. 8 (2020) 1-10) developed a calorimeter-based detector that can be used for real-time measurements. However, the detector does not have the capability to be utilized during irradiations. Because all the proton energy is deposited within the calorimeter, it is not suitable for use during treatment as no energy would be deposited within the treatment site. An additional study by Chen et. Al. (Investigation of YAG:Ce-Based Optical Fibre Sensor for Use in Ultra-Fast External Beam Radiotherapy Dosimetry, J. Light. Technol. 37 (2019) 4741-4747) shows an inorganic scintillating fiber can distinguish individual beam pulses. This device, however, also lacks real-time beam control.

### Dosimeter Overview

As discussed above, a major hurdle for FLASH-RT is the ability to monitor and control the delivery of radiation pulses in-situ. The present disclosure provides a stand-alone dosimeter for use in online beam monitoring and control with an adequate resolution and response time to mitigate the potential risks of over-dose delivery. The disclosed dosimeter provides three major operations: scintillation, signal transmission, and electronics/acquisition.

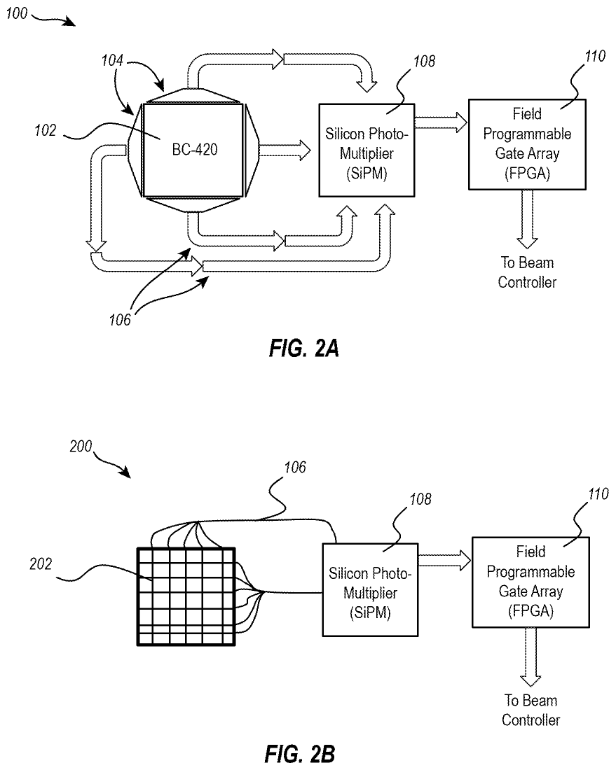

FIG. 2A is a schematic of an example dosimeter 100. The dosimeter 100 includes a scintillator 102 (shown here as a BC-420 scintillator) that is optically coupled to one or more fiberoptic lines 106 (e.g., via one or more light guides 104). As shown, the fiberoptic lines 106 are also optically connected to a photomultiplier 108 (shown here as a silicon photomultiplier “SiPM”). The photomultiplier 108 is communicatively coupled to a controller 110 (shown here as a field programmable gate array “FPGA”). The controller 110 is communicatively coupled to the beam controller and can therefore control the beam in real time. For example, the controller 110 can be configured with a “kill switch” that stops the beam if a threshold dose and/or dosage rate is reached.

The dosimeter 100 can also include a converter (not shown), which functions as a preamplifer and signal converter from the photomultiplier 108 to the controller 110. This enables control and adjustment of the bias voltage of the photomultiplier 108, for example, and also provides an additional differential amplifier across all independent channels.

In use, the proton beam interacts substantially orthogonally with the scintillator 102, and the fiberoptic lines 106 function to transfer signals away from the treatment gantry (e.g., into an adjacent room) where the photomultiplier 108 resides. The photomultiplier 108 functions to convert the light signals into electronic signals, and then sends the electronic signals to the controller 110 (e.g., a custom programmed FPGA).

While the example dosimeter 100 of FIG. 2A shows a dosimeter 100 with a flat sheet/plate design, FIG. 2B illustrates another example dosimeter 200 that includes a scintillator 202 comprising scintillating fiberoptic lines rather than a flat sheet/plate construction. The dosimeter 200 may otherwise be similar to dosimeter 100. Except where specified otherwise, the present disclosure is generic to both dosimeter 100 and dosimeter 200.

### Example Scintillation Materials

In contrast to prior approaches, the presently disclosed dosimeter 100 utilizes stand-alone scintillation detectors comprising fast organic materials. The fast response times of organic scintillators on the order of a few nanoseconds enable the detector to respond on similar timescales to the beam pulses, eliminating delayed signals and potential error. The near tissue equivalence of such organics is also beneficial, allowing for a natural translation to absorbed dose within the patient. While it is understood that organics, in general, have a lower light yield than inorganics, the high dose rates of FLASH counteract this issue and can even beneficially reduce saturation effects of the dosimeter.

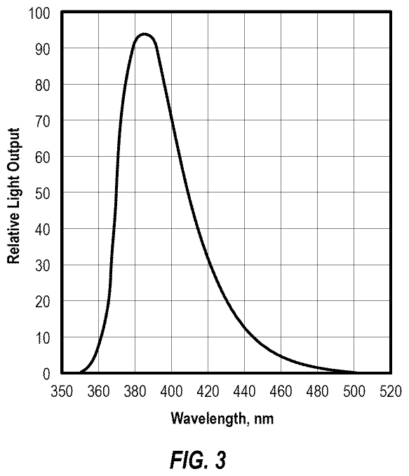

An example scintillator material useful in the disclosed dosimeter is BC-420, available from Saint-Gobain Crystals, Hiram, Ohio. The BC-420 material maintains a fast decay time of approximately 1.3 ns, making it ideal for fast-timing applications such as FLASH-RT. The emission spectrum for BC-420 is shown in FIG. 3. Additional properties of BC-420 are provided in Table 1 located below.

TABLE 1
Properties of BC-420 Scintillator Material

| Scintillation Properties | Physical Properties |
|---|---|
| 0.5 ns Rise Time | 1.032 g/cc |
| 1.5 ns Decay Time | 1.58 refractive index |
| 391 nm Max Emission (350-500) | |
| 64% Anthracene | |
| (~11,100 photons/MeV) | |

The emission spectrum shows a max emission or approximately 390 nm. This also beneficially corresponds to the absorption spectrum of the preferred photomultipliers, as discussed in more detail below. The BC-420 material also has a density with good tissue equivalence, with a density of approximately 1.032 g/cc.

The disclosed dosimeter 100 can additionally or alternatively include other scintillator materials. The scintillator materials are preferably based on organic emitter molecules (i.e., fluors). The base of the scintillator material can include an aromatic plastic, such as polyvinyltoluene (PVT) or polystyrene (PS), or another plastic with suitable transparency and mechanical properties, such as polymethylmethacrylate (PMMA). Suitable fluors for inclusion in the base include polyphenyl hydrocarbons, oxazole and oxadiazole aryls, for example.

In some embodiments, the scintillator materials included in the dosimeter 100 have one or more properties similar to those shown in Table 1 for BC-420. For example, the scintillator materials may provide a rise time, decay time, emission profile, emission output, density, and/or refractive index within ±10% of the value(s) shown in Table 1, or within ±7.5% of the value(s) shown in Table 1, or within ±5% of the value(s) shown in Table 1, or within ±2.5% of the value(s) shown in Table 1.

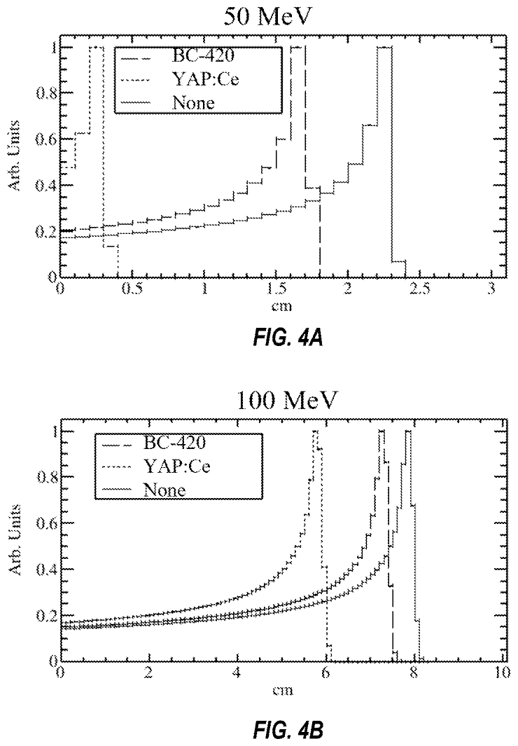

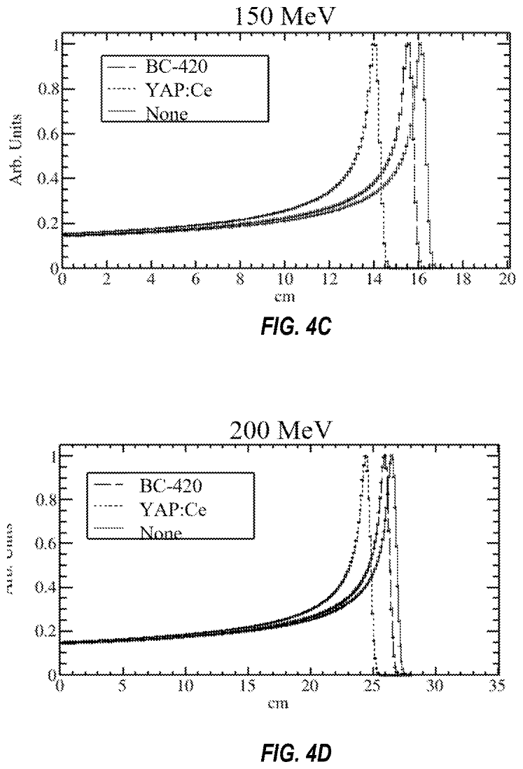

In comparison to an inorganic material such as Cerium doped Yttrium Aluminum Perovskite (YAP:Ce), which has a comparable decay time, BC-420 allows for sufficient energy deposition from protons while not significantly altering the beam after detection. By detecting the particles, there is an inherent change in the beam characteristics ultimately delivered to the patient from those produced directly from the gantry. The BC-420 material has displayed a smaller shift in the peak location, thus more suitable than that of YAP:Ce. Simulated results are shown in FIGS. 4A to 4D, with a simulated scintillator thickness of 5 mm. Four separate thickness were simulated (0.5 mm, 1 mm, 5 mm, 1 cm), all producing similar patterns, in each case the organic BC-420 displaying a smaller shift in Bragg peak location in comparison to the inorganic YAP:Ce.

In some embodiments, the scintillator 102 comprises a scintillator material with a sheet shape, preferably having a thickness of 1 mm to 5 mm. Too great a thickness in the scintillator will negatively impact the characteristics of the beam after detection. Excessive thickness will strongly filter the energies of protons such that the compensation required to achieve the depth of the tumor would lead to, in some cases, significant error in the overall dose distribution. In contrast, however, scintillators with too little volume may not provide a significant signal to have an acceptably low error in measurement. A scintillator thickness of 1 mm to 5 mm beneficially provides low energy filtering as well as sufficient interaction to provide adequate signals.

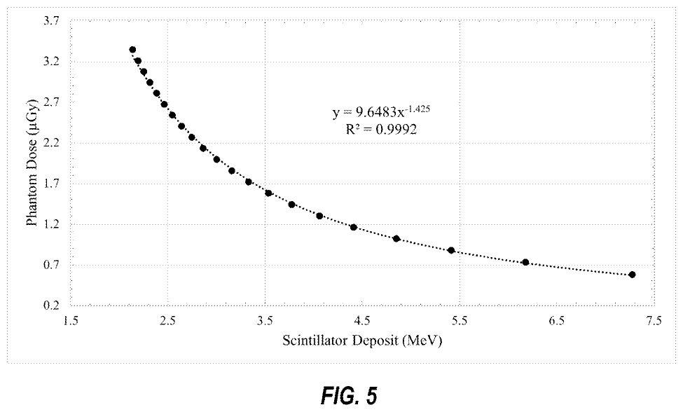

FIG. 5 shows a well-defined response between scintillator energy deposit and the total dose to a tissue phantom. The delivered dose as a function of scintillator energy deposit is shown per primary simulated particle after interaction with a 5 mm BC-420 scintillator.

In other embodiments, the scintillator 202 can comprise a plurality of scintillating fiberoptic lines. Such scintillating fiberoptic lines may comprise any of the same scintillator materials described above. In such embodiments, the scintillating fiberoptic lines can be directly coupled to the fiberoptic lines 106 (e.g., each scintillating fiberoptic line can be connected to a corresponding fiberoptic line 106. Cross-hatching of the scintillating fiberoptic lines is also utilized in some embodiments. For example, multiple layers of scintillating fiberoptic lines may be stacked together, with one or more of the layers orienting their scintillating fiberoptic lines transverse to one or more other layers.

### Scintillation Signal Transmission

In some embodiments, fiberoptic lines 106 are utilized for transmission of the light signals generated by the scintillator. The fiberoptic lines 106 beneficially maintain the temporal characteristics of the detector, as the transmission time from scintillator to the photomultiplier is significantly small (below 10 ns) for up to approximately 1.9 meters of fiberoptic line. The fiberoptic lines 106 also beneficially separate electronics components (e.g., photomultiplier 106, converter, and/or controller 110) from the radiation fields near the treatment gantry. In some embodiments, the fiberoptic lines 106 enable the scintillator and the electronics components to be separated by 10 cm or more, or by 20 cm or more, or by 30 cm or more, or by 45 cm or more, or by 60 cm or more, or by 90 cm or more, or by 120 cm or more, or by 150 cm or more, or by 180 cm or more, or a range with any combination of the foregoing as endpoints.

Transferring the generated light signals away from the scintillator 102 beneficially minimizes the interaction of the beam with electronics components of the detector apart from the scintillator 102. The fiberoptic lines 106 are also arranged so as to send the generated light signals out the sides of the scintillator 102, rather than the front or back. The fiberoptic lines 106 are thus coupled to the edges of the scintillator 102. As shown in FIG. 2, the system 100 may include light guides 104 to direct signals form the edges of the scintillator 102 to the fiberoptic lines 106. Additionally, or alternatively, fiberoptic lines 106 can be directly coupled to or embedded in the scintillator 102.

### Photomultiplier

The photomultiplier 108 is used to convert the photons to electrical signals. In a preferred embodiment, the photomultiplier 108 is a silicon photomultiplier (SiPM). SiPMs have a major advantage over other common photomultiplier tubes in that the signal conversion from light input to photoelectron production is generally well below 1 ns, allowing for rapid transmission from signal production to electronics. SiPM's can also be small and compact, and only require a small bias voltage (e.g., typically around 54 V) and provide a route for simultaneous readout across multiple channels. A SiPM could thus read multiple fiberoptic lines 106 while still maintaining its adequate response time. This is advantageous in enhancing the signals produced from the scintillator 102 and therefore reducing the uncertainty in dose measurements.

### Controller

The controller 110 may include any computer system comprising one or more processors and one or more hardware storage devices (i.e., memory) executable by the one or more processors to enable the functionality described herein. In some embodiments, the controller 110 comprises an FPGA. The controller preferably has a sampling rate that provides an effective temporal resolution. In one embodiment, an FPGA with a sampling rate of 80 MHz provides a limit of temporal resolution of 12.5 ns. This FPGA has been tested to show that signals can be produced after some predetermined threshold has been reached within 1 clock cycle (12.5 ns). In some embodiments, the controller 110 has a time resolution of no greater than 100 ns, or no greater than 90 ns, or no greater than 80 ns, or no greater than 70 ns, or no greater than 60 ns, or no greater than 50 ns, or no greater than 40 ns, or no greater than 30 ns, or no greater than 20 ns, or no greater than 15 ns, such as about 12.5 ns or less, or a range with any combination of the foregoing as endpoints.

With such time resolution, the controller 110 will have sufficient time to monitor the beam and stop treatment should a dose limit or rate be reached, or other error scenarios arise. The known sampling rate along with real-time beam monitoring can also enable the tracking of any extraneous dose delivered after a beam-off signal may be received.

In operation, once a threshold or unexpected/error circumstance is encountered, a signal can be sent from the controller 110 indicating a beam off state while continually monitoring the dose delivered during the treatment. This allow for effective measurement of the total dose delivered to the patient and amount of potential overshoot from the anticipated treatment.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be omitted or essentially omitted from the disclosed embodiments. For example, optionally, scintillator materials not specifically disclosed herein, and/or electronics components not specifically disclosed herein may optionally be omitted from the dosimeter.

An embodiment that “essentially omits” or is “essentially free of” a component may include trace amounts and/or non-functional amounts of the component. For example, an “essentially omitted” component may be included in an amount no more than 10%, no more than 5%, no more than 2.5%, no more than 1%, no more than 0.1%, or no more than 0.01% by total weight of the relevant composition (e.g., the scintillator material). This is likewise applicable to other negative modifier phrases such as, but not limited to, “essentially omits,” “essentially without,” similar phrases using “substantially” or other synonyms of “essentially,” and the like.

1. A dosimeter configured for use in FLASH radiotherapy (FLASH-RT), the dosimeter comprising:
a scintillator configured for positioning within a beam path of a proton beam generator;
a plurality of fiberoptic lines each having a first end and a second end, the first ends being optically coupled to the scintillator;
a photomultiplier to which the second ends of the plurality of fiberoptic lines are optically coupled; and
a controller to which the photomultiplier is communicatively coupled, the controller being configured for communication with a beam controller of the proton beam generator.

2. The dosimeter of claim 1, wherein the scintillator comprises a plurality of scintillating fiberoptic lines.

3. The dosimeter of claim 2, wherein the plurality of scintillating fiberoptic lines are arranged in layered, cross-hatched configuration.

4. The dosimeter of claim 2, wherein each scintillating fiberoptic line is coupled to a separate fiberoptic line.

5. The dosimeter of claim 2, wherein the scintillator comprises a flat sheet.

6. The dosimeter of claim 5, wherein the flat sheet has a thickness of 1 mm to 5 mm.

7. The dosimeter of claim 1, wherein the fiberoptic lines are coupled to a side edge of the scintillator and extend therefrom.

8. The dosimeter of claim 1, further comprising one or more light guides for transmitting light signals from the scintillator to the fiberoptic lines.

9. The dosimeter of claim 1, further comprising a converter communicatively coupled to the photomultiplier and the controller and configured to adjust a bias voltage of the photomultiplier.

10. The dosimeter of claim 9, wherein the converter is further configured to function as an amplifier.

11. The dosimeter of claim 1, wherein the scintillator comprises a BC-420 scintillator material.

12. The dosimeter of claim 1, wherein the scintillator comprises a scintillator material that provides one or more of (within ±10%):
a rise time of 0.5 ns;
a decay time of 1.5 ns;
a maximum emission peak of 391 nm;
an emission level of 64% anthracene;
a density of 1.032 g/cc; or
a refractive index of 1.58.

13. The dosimeter of claim 1, wherein the scintillator comprises an aromatic plastic.

14. The dosimeter of claim 13, wherein the aromatic plastic comprises polyvinyltoluene (PVT) and/or polystyrene (PS).

15. The dosimeter of claim 1, wherein the scintillator and the photomultiplier are separated by 10 cm or more.

16. The dosimeter of claim 1, wherein the photomultiplier comprises a silicon photomultiplier (SiPM).

17. The dosimeter of claim 1, wherein the controller comprises a field programmable gate array (FPGA).

18. A FLASH-RT system, comprising:
a proton beam generator; and
a dosimeter as in claim 1 disposed within a beam path of the proton beam generator.

19. A dosimeter configured for use in FLASH radiotherapy (FLASH-RT), the dosimeter comprising:
a scintillator configured for positioning within a beam path of a proton beam generator, wherein the scintillator comprises a BC-420 scintillator material;
a plurality of fiberoptic lines each having a first end and a second end, the first ends being optically coupled to the scintillator, wherein the fiberoptic lines are coupled to a side edge of the scintillator and extend therefrom;
a photomultiplier to which the second ends of the plurality of fiberoptic lines are optically coupled, wherein the photomultiplier comprises a silicon photomultiplier (SiPM); and
a controller to which the photomultiplier is communicatively coupled, the controller being configured for communication with a beam controller of the proton beam generator, wherein the controller comprises a field programmable gate array (FPGA).

20. A FLASH-RT system, comprising:
a proton beam generator; and
a dosimeter as in claim 19 disposed within a beam path of the proton beam generator.
